(19) United States
(12) Patent Application Publication
(10) Pub. No.: US 2026/0218120 A1
(43) Pub. Date: Jul. 30, 2026

(54) SALT-TOLERANT MANGANESE-OXIDIZING BACTERIAL STRAIN YJT-1 AND APPLICATION THEREOF

(71) Applicant: Qingdao University of Technology, Qingdao (CN)

(72) Inventors: Xueqin WANG, Qingdao (CN); Huawei WANG, Qingdao (CN); Yanan WANG, Qingdao (CN); Fei WANG, Qingdao (CN); Mingxuan BAI, Qingdao (CN); Yingjie SUN, Qingdao (CN); Xinlei DUAN, Qingdao (CN); Yifan ZHENG, Qingdao (CN); Xiongbo CUI, Qingdao (CN); Yuxin LIU, Qingdao (CN); Huimin LI, Qingdao (CN); Xinhui LI, Qingdao (CN); Rongwen LIU, Qingdao (CN); Jianwei ZHAO, Qingdao (CN); Jing SONG, Qingdao (CN); Xiaotong CHEN, Qingdao (CN); Jiahui YI, Qingdao (CN)

(21) Appl. No.: 19/643,936

(22) Filed: Apr. 10, 2026

(30) Foreign Application Priority Data
Nov. 24, 2025 (CN) 202511725623.X

(51) Int. Cl. C12N 1/205; B09C 1/10; C02F 3/34; C12P 3/00; C02F 101/32; C12R 1/01

(52) U.S. Cl. CPC C12N 1/205; B09C 1/10; C02F 3/34; C12P 3/00; C02F 2101/327; C12R 2001/01

(57) ABSTRACT

Asalt-tolerant manganese-oxidizing bacterial strain YJT-1, which is a Halobacillus sp. strain, and has been deposited in China General Microbiological Culture Collection Center (CGMCC) on March 7, 2025 with an accession number of CGMCC No. 33748. This Halobacillus sp. strain possesses excellent manganese-oxidizing capability and salinity tolerance, and can effectively induce the formation of biogenic manganese oxide to achieve the degradation of BaP and other polycyclic aromatic hydrocarbons. This application also provides an application of the Halobacillus sp. strain in the remediation of organic pollution in high-salinity water bodies or soil.

### REFERENCE TO AN ELECTRONIC SEQUENCE LISTING

The contents of the electronic sequence listing (SequenceListing.xml; Size: 3,416 bytes; and Date of Creation: March 31, 2026) are herein incorporated by reference in its entirety.

### CROSS-REFERENCE TO RELATED APPLICATIONS

This application claims the benefit of priority from Chinese Patent Application No. 202511725623.X, filed on November 24, 2025. The content of the aforementioned application, including any intervening amendments thereto, is incorporated herein by reference in its entirety.

### TECHNICAL FIELD

This application relates to microbial biotechnology, and more particularly to a salt-tolerant manganese-oxidizing bacterial strain YJT-1 and an application thereof.

### BACKGROUND

Salinization of soil or water pose a serious threat to agriculture and ecology. At the same time, the accumulation of persistent organic pollutants (e.g., polycyclic aromatic hydrocarbons (PAHs)) in saline-alkali soil or water is increasingly prominent. Many high-molecular-weight polycyclic aromatic hydrocarbons (such as benzo[a]pyrene (BaP)) are difficult to degrade effectively by abiotic or biological means. After entering the saline-alkaline soil environment, benzo[a]pyrene is prone to adsorbing strongly with organic matter and mineral surfaces in soil particles, leading to its enrichment in the solid phase. Traditional physical and chemical remediation methods, such as thermal desorption and persulfate activation, can remove PAHs, but there are some problems such as high energy consumption, reagent residue and soil organic matter loss, which are not suitable for the sustainable remediation practice of large-scale farmland. Therefore, the strategy of in situ bioremediation with microorganisms as the core has received increasing attention.

Manganese-oxidizing microorganisms can convert divalent manganese into high-valent manganese oxide through metabolism. High-valent biogenic manganese oxides have high reactivity and can perform non-specific oxidation of aromatic compounds, thus promoting the transformation and mineralization of persistent organic pollutants. At the same time, the active intermediates generated during the manganese oxidation process can also improve the degradation efficiency of high molecular weight polycyclic aromatic hydrocarbons such as BaP. However, there are obvious limitations in the direct application of common manganese-oxidizing bacteria in saline-alkali soils. High saline-alkali conditions can cause osmotic stress and ion toxicity, inhibit the normal life activities of microorganisms and their manganese oxidation ability, and lead to a decline in remediation functions. Therefore, the screening and cultivation of functional strains that can not only tolerate salt-alkali stress, maintain robust growth conditions, but also efficiently exhibit manganese oxidation capability and degrade organic pollutants like benzo[a]pyrene, has become a crucial approach to deal with the combined pollution of salinization and polycyclic aromatic hydrocarbons.

### SUMMARY

An object of the present disclosure is to provide a salt-tolerant manganese-oxidizing bacterial strain YJT-1 and an application thereof. The bacterial strain YJT-1 can effectively degrade organic pollutants, such as benzo[a]pyrene.

The following technical solutions of the present disclosure are adopted.

A salt-tolerant manganese-oxidizing bacterial strain YJT-1, wherein the bacterial strain YJT-1 is Halobacillus sp., which has been deposited in China General Microbiological Culture Collection Center (CGMCC) on March 7, 2025 with an accession number of CGMCC No. 33748.

In some embodiments, this disclosure provides an application of the aforementioned bacterial strain YJT-1 in degradation of benzo(a)pyrene, degradation of benzo(a)pyrene under a high-salinity and/or high-manganese condition, oxidation of divalent manganese to induce formation of biogenic manganese oxides under a high-salinity condition, remediation of benzo[a]pyrene-contaminated soil or water, or preparation of a microbial agent for remediating benzo[a]pyrene-contaminated soil or water.

In some embodiments, this disclosure provides a method of remediating a benzo[a]pyrene-contaminated soil sample or a benzo[a]pyrene-contaminated water sample, comprising:

introducing a bacterial suspension of the aforementioned bacterial strain YJT-1 into the benzo[a]pyrene-contaminated soil sample or the benzo[a]pyrene-contaminated water sample to degrade benzo[a]pyrene.

In some embodiments, the benzo[a]pyrene-contaminated soil sample is a benzo[a]pyrene-contaminated high-salinity soil sample, a benzo[a]pyrene-contaminated high-manganese soil sample or a combination thereof.

In some embodiments, the present disclosure provides a method for preparing a biogenic manganese oxide, comprising:

oxidizing divalent manganese ions under the action of the aforementioned bacterial strain YJT-1 to form the biogenic manganese oxide.

In some embodiments, the present disclosure provides a microbial agent for remediating a benzo[a]pyrene-contaminated soil or water sample, comprising:

cells of the aforementioned bacterial strain YJT-1.

The bacterial strain YJT-1 provided herein possesses excellent manganese-oxidizing capability and salinity tolerance, and can effectively induce the formation of biogenic manganese oxide to achieve the degradation of BaP and other polycyclic aromatic hydrocarbons. This confers significant application potential for the in-situ remediation of organic pollution in high-salinity water bodies or soils.

### BRIEF DESCRIPTION OF THE DRAWINGS

FIG. 1 is a culture photograph of a bacterial strain YJT-1 according to an embodiment of the present disclosure;

FIG. 2 is a scanning electron microscopy (SEM) image of the bacterial strain YJT-1 according to an embodiment of the present disclosure;

FIG. 3 is a phylogenetic tree of the bacterial strain YJT-1;

FIG. 4 shows a biogenic manganese oxide (BMO) content produced by the bacterial strain YJT-1under different salinity levels;

FIG. 5 shows a BMO content produced by the bacterial strain YJT-1 under different pH conditions;

FIG. 6 shows a BMO content produced by the bacterial strain YJT-1 at different Mn(II) concentrations;

FIG. 7 shows a BMO content produced by the bacterial strain YJT-1 with different carbon sources;

FIG. 8 shows a degradation efficiency of benzo[a]pyrene (BaP) by the bacterial strain YJT-1 in a high-salinity water sample;

FIG. 9 shows a degradation efficiency of BaP in Xinjiang saline-alkali soil by the bacterial strainYJT-1;

FIG. 10 shows a degradation efficiency of BaP in Ningxia saline-alkali soil by the bacterial strain YJT-1; and

FIG. 11 shows a degradation efficiency of BaP in Shandong saline-alkali soil by the bacterial strain YJT-1.

### DETAILED DESCRIPTION OF EMBODIMENTS

For terms not specifically defined herein, they can be given the meaning that those skilled in the art can give them, according to the disclosure content and context.

The present disclosure will be further illustrated in detail with reference to the embodiments. It should be noted that the embodiments are merely illustrative, and are not intended to limit the present disclosure.

Unless otherwise specified, the experimental materials, reagents or drugs in the following embodiments are all commercially available.

### EMBODIMENT 1

A method of obtaining a bacterial strain YJT-1was performed through the following steps.

### (S1) Sampling

Soil samples were collected from a saline-alkali site in Dongying City. A contaminated area in the saline-alkali site was divided into a plurality of blocks. Within each of the plurality of blocks, an equal amount of soil was randomly sampled, thoroughly mixed and separated from impurities such as stones, tiles, and plant tubers. These soil samples were then ground using a crusher and sieved through a 60-mesh screen to further remove coarse particles like gravel and plant residues. Steps (S2-S4) were performed within 24 h on all processed soil samples, so as to isolate the bacterial strain YJT-1.

### (S2) Enrichment

5.0 g of the processed soil samples was added to 100 mL of sterile water and incubated at 30°C with shaking at 120 rpm/min for 8 h, followed by centrifugation to give a supernatant. 6.0 mL of the supernatant was transferred into an Erlenmeyer flask (500 mL) containing 300 mL of inorganic salt liquid culture medium and cultured at 30°C with shaking at 120 rpm/min for 7 days, so as to obtain a first culture. Then, 6.0 mL of the first culture was subcultured into another inorganic salt liquid culture medium and incubated again under the same conditions (30°C, 120 rpm/min) for another 7 days, so as to obtain a second culture. This subculturing process was performed for three successive rounds, so as to obtain a third culture after a third round, that is, a high-concentration bacterial broth.

The inorganic salt liquid culture medium contained 10 g/L NaCl, 0.4 g/L MgCl₂·6H₂O, 0.754 g/L CaCl₂, 5 g/L KCl, 0.142 g/L Na₂SO₄, 0.085 g/L NaNO₃, 0.026 g/L K₂HPO₄, 0.2 g/L MnSO₄·H₂O, 0.8 g/L peptone, and 0.2 g/L yeast extract.

### (S3) Isolation and purification

The high-concentration bacterial broth was serially diluted to concentrations of 10⁻¹, 10⁻², 10⁻³, 10⁻⁴, 10⁻⁵, 10⁻⁶, 10⁻⁷, and 10⁻⁸. Aliquots of each dilution were spread onto inorganic salt agar plates and evenly distributed using a sterile spreader. These plates were incubated at 30°C with shaking at 120 rpm/min for 4 days in a constant temperature shaking incubator. After well-isolated single colonies appeared uniformly on these plates, individual colonies with distinct morphological characteristics were picked using an inoculation loop. Each colony was streaked onto a fresh inorganic salt agar plate for purification. This streaking process was repeated until colonies with uniform morphology were obtained, then yielding to-be-screened strains.

The inorganic salt agar plates each contained 10 g/L NaCl, 0.4 g/L MgCl₂·6H₂O, 0.754 g/L CaCl₂, 5 g/L KCl, 0.142 g/L Na₂SO₄, 0.085 g/L NaNO₃, 0.026 g/L K₂HPO₄, 0.2 g/L MnSO₄·H₂O, 0.8 g/L peptone, 0.2 g/L yeast extract, and 2 g/L agar.

### (S4) Acclimatization

The to-be-screened strains were respectively inoculated at 2% (v/v) into Erlenmeyer flasks (500 mL) containing 300 mL of high-salinity inorganic salt liquid culture mediums and subjected to shock exposure. The culture mediums were incubated at 30°C with shaking at 120 rpm/min for 7 days. The content of biogenic manganese oxides (BMOs) in the culture was determined using a leucoberbelin blue (LBB) assay to evaluate the manganese-oxidizing capability of the to-be-screened strains. The results indicated that a strain with a serial number of YJT-1 was identified as exhibiting strong manganese-oxidizing activity under high-salinity conditions. At a salinity of 3.5% and an initial Mn(II) concentration of 100 mg/L, a BMO content of 74.21 mg/L was produced by the bacterial strain YJT-1. The manganese-oxidizing strain (namely, the bacterial strain YJT-1, obtained through the screening process of this application) was then inoculated into glycerol stock tubes and stored at -80°C.

The high-salinity inorganic salt liquid culture mediums each contained 50 g/L NaCl, 0.4 g/L MgCl₂·6H₂O, 0.754 g/L CaCl₂, 5 g/L KCl, 0.142 g/L Na₂SO₄, 0.085 g/L NaNO₃, 0.026 g/L K₂HPO₄, 0.2 g/L MnSO₄·H₂O, 0.8 g/L peptone, and 0.2 g/L yeast extract.

### EMBODIMENT 2

Morphological identification of the salt-tolerant manganese-oxidizing bacterial strain YJT-1, and sequence analysis of 16S rDNA were performed.

### (1) Morphological characteristics

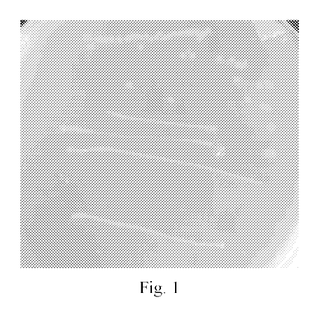

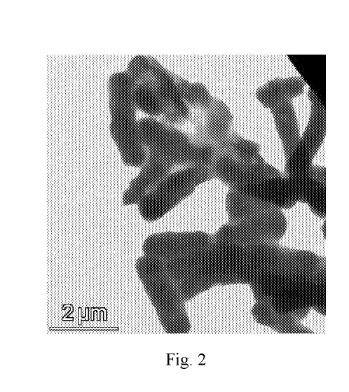

Morphological characteristics were shown in FIG. 1. The bacterial strain YJT-1 was incubated on an inorganic salt agar plate at 30°C for 4 days in a constant temperature incubator. Circular, pale yellow colonies with smooth edges were observed. The colonies were tightly adhered to the agar plate, and were opaque. A scanning electron microscopy (SEM) image was shown in FIG. 2. The cells were observed to be rod-shaped, with well-defined edges, parallel sides, bluntly rounded ends, and a diameter of approximately 2.5 μm.

### (2) Polymerase chain reaction (PCR) amplification and sequence analysis of 16S rDNA gene of the bacterial strain YJT-1

Genomic DNA extraction, PCR amplification and sequence analysis were performed by Sangon Biotech (Shanghai) Co., Ltd. The 16S rDNA sequence of the bacterial strain YJT-1was 1,448 bp in length. The complete DNA sequence was listed in the Sequence Listing as SEQ ID NO: 1.

The SEQ ID NO: 1 was shown as follows:

ggcctgtctgcagtcgagcgcgggaagcgagctgatcccttcggggtgacgctcgtggaagagcggcggacgggtgagtaacacgtgggcaacctgcctgtaagatcgggataactccgggaaaccggggctaataccgggtaatactttctttcgcatgaaggaaagttgaaagatggttctcgctatcacttacagatgggcccgcggcgcattagctagttggtgaggtaacggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgaacgatgaaggtcttcggatcgtaaagttctgttgttagggaagaacaagtaccgtgcgaatagagcggtaccttgacggtacctaacgaggaagccccggctaactacgtgccagcagccgcggtaatacgtagggggcaagcgttgtccggaattattgggcgtaaagcgcgcgcaggcggttccttaagtctgatgtgaaagcccacggctcaaccgtggagggtcattggaaactggggaacttgaggacagaagaggagagtggaattccacgtgtagcggtgaaatgcgtagatatgtggaggaacaccagtggcgaaggcgactctctggtctgtttctgacgctgaggtgcgaaagcgtgggtagcaaacaggattagataccctggtagtccacgccgtaaacgatgagtgctaggtgttagggggcttccaccccttagtgctgaagttaacgcattaagcactccgcctggggagtacggccgcaaggctgaaactcaaaggaattgacgggggcccgcacaagcggtggagcatgtggtttaattcgaagcaacgcgaagaaccttaccaggtcttgacatccttggacctccctagagatagggatttcccttcggggaccaagtgacaggtggtgcatggttgtcgtcagctcgtgtcgtgagatgttgggttaagtcccgcaacgagcgcaacccctgatcttagttgccagcattcagttgggcactctaaggtgactgccggtgacaaaccggaggaaggcggggatgacgtcaaatcatcatgccccttatgacctgggctacacacgtgctacaatggatggtacaaagggcagcgaagccgcgaggtgtagcaaatcccataaaaccattctcagttcggattgcaggctgcaactcgcctgcatgaagccggaatcgctagtaatcgcggatcagcatgccgcggtgaatacgttcccgggccttgtacacaccgcccgtcacaccacgagagttggtaacacccgaagtcggtgaggtaacccttaggagcaccgccccaacccgcccc.

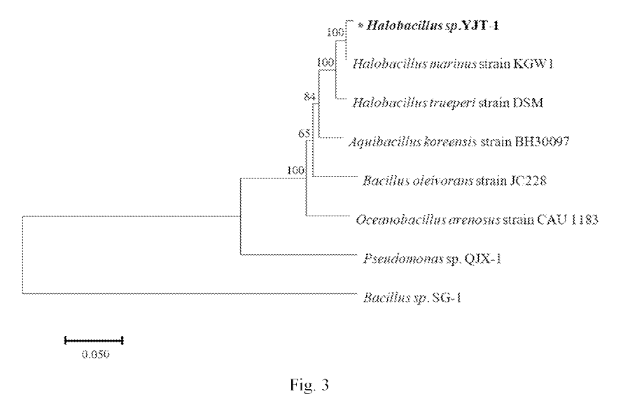

Sequence comparison with the GenBank database was conducted using BLAST software. The results showed that the 16S rDNA sequence of the bacterial strain YJT-1 exhibited the highest similarity to Halobacillus marinus strain KGW1. Therefore, the bacterial strain YJT-1 was identified as a salt-tolerant, spore-forming bacterium belonging to the genus Halobacillus. It was taxonomically designated as Halobacillus sp. YJT-1. A phylogenetic tree of the bacterial strain YJT-1 was presented in FIG. 3.

### EMBODIMENT 3

In this embodiment, the BMO synthesis ability of the bacterial strain YJT-1 was tested under different salinities.

Four Erlenmeyer flasks (500 mL) were prepared, each containing 200 mL of basal inorganic salt liquid culture medium. The salinity of each culture medium was adjusted to 1%, 3.5%, 5% and 10%, respectively. The Mn(II) concentration in each culture medium was set at 100 mg/L. Peptone (0.8 g/L) and yeast extract (0.2 g/L) were added to each culture medium as a carbon source. A culture of the bacterial strain YJT-1 in a logarithmic growth phase, which was obtained from step (S4), was inoculated into each sterile medium at 2% (v/v). These Erlenmeyer flasks were incubated at 30°C with shaking at 120 rpm/min for 20 consecutive days. 5 mL of the culture was collected from each Erlenmeyer flask every 4 days, followed by centrifugation, so as to retain precipitates. The BMO content in the precipitates was quantified using the LBB assay. The manganese-oxidizing capability of the bacterial strain YJT-1 was evaluated based on variation in the BMO content.

Each basal inorganic salt liquid culture medium contained 0.4 g/L MgCl₂·6H₂O, 0.754 g/L CaCl₂, 5 g/L KCl, 0.142 g/L Na₂SO₄, 0.085 g/L NaNO₃, and 0.026 g/L K₂HPO₄.

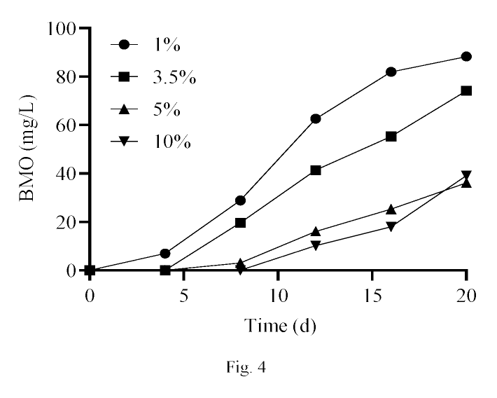

As shown in FIG. 4, with the extension of the culture time, the BMO content under all salinity conditions gradually increased, thereby demonstrating the sustained manganese-oxidizing capability of the bacterial strainYJT-1. On the 20th day, the BMO content was the highest at a salinity of 1%, reaching 88.27 mg/L. Secondly, the BMO content was 74.21 mg/L at a salinity of 3.5%. Under the condition of 10% salinity, the bacterial strain YJT-1 produced 38.59 mg/L of BMO, indicating that in a high-salt environment, the bacterial strainYJT-1 still continuously produced BMO.

### EMBODIMENT 4

In this embodiment, the BMO synthesis ability of the bacterial strain YJT-1 was tested under different pH conditions.

Four Erlenmeyer flasks (500 mL) were prepared, each containing 200 mL of basal inorganic salt liquid culture medium. The pH of each culture medium was adjusted to 6, 6.5, 7.2 and 7.6, respectively. The salinity in each culture medium was set at 3.5%. The Mn(II) concentration in each culture medium was set at 100 mg/L. Peptone (0.8 g/L) and yeast extract (0.2 g/L) were added to each culture medium as a carbon source. The culture of the bacterial strain YJT-1 in the logarithmic growth phase, which was obtained from step (S4), was inoculated into each sterile medium at 2% (v/v). These Erlenmeyer flasks were incubated at 30°C with shaking at 120 rpm/min for 20 consecutive days. 5 mL of the culture was collected from each Erlenmeyer flask every 4 days, followed by centrifugation, so as to retain precipitates. The BMO content in the precipitates was quantified using the LBB assay. The manganese-oxidizing capability of the bacterial strain YJT-1 was evaluated based on changes in the BMO content.

Each basal inorganic salt liquid culture medium contained 0.4 g/L MgCl₂·6H₂O, 0.754 g/L CaCl₂, 5 g/L KCl, 0.142 g/L Na₂SO₄, 0.085 g/L NaNO₃, and 0.026 g/L K₂HPO₄.

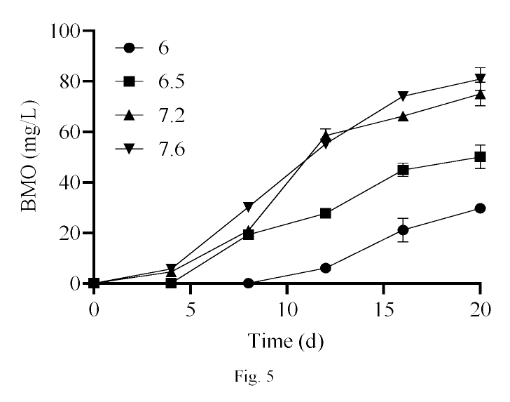

As shown in FIG. 5, when the pH value of the culture system was controlled within a range of 7.0-8.0, especially 7.2-7.6, the bacterial strain YJT-1 exhibited superior BMO synthesis capability. At pH 7.6, the BMO content reached 80.87 mg/L after 20 days of culture, which was significantly higher than that under acidic conditions (29.69 mg/L at pH 6.0) and weakly acidic conditions (50.16 mg/L at pH 6.5). Compared with the condition of pH 6.0, the BMO content at pH 7.6 was approximately 2.72 times that at pH 6.0, which demonstrated that the manganese-oxidizing activity of the bacterial strain YJT-1 was significantly promoted in such a weakly alkaline environment.

### EMBODIMENT 5

In this embodiment, the BMO synthesis ability of the bacterial strain YJT-1 was tested at different Mn(II) concentrations.

Five Erlenmeyer flasks (500 mL) were prepared, each containing 200 mL of basal inorganic salt liquid culture medium. The Mn(II) concentration of each culture medium was adjusted to 5 mg/L, 10 mg/L, 25 mg/L, 50 mg/L, and 100 mg/L, respectively. The salinity in each culture medium was set at 3.5%. Peptone (0.8 g/L) and yeast extract (0.2 g/L) were added to each culture medium as a carbon source. The culture of the bacterial strain YJT-1 in the logarithmic growth phase, which was obtained from step (S4), was inoculated into each sterile medium at 2% (v/v). These Erlenmeyer flasks were incubated at 30°C with shaking at 120 rpm/min for 20 consecutive days. 5 mL of the culture was collected from each Erlenmeyer flask every 4 days, followed by centrifugation, so as to retain precipitates. The BMO content in the precipitates was quantified using the LBB assay. The manganese-oxidizing capability of the bacterial strain YJT-1 was evaluated based on variation in the BMO content.

Each basal inorganic salt liquid culture medium contained 0.4 g/L MgCl₂·6H₂O, 0.754 g/L CaCl₂, 5 g/L KCl, 0.142 g/L Na₂SO₄, 0.085 g/L NaNO₃, and 0.026 g/L K₂HPO₄.

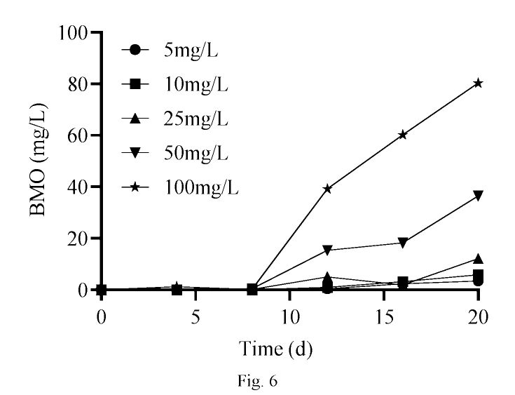

As shown in FIG. 6, the BMO content significantly increased with the increase of Mn(II) concentration. When the Mn(II) concentration was 100 mg/L, the BMO content was the highest, reaching 80.33 mg/L. When the Mn(II) concentration was 5 mg/L, the BMO content was the lowest, at 3.47 mg/L. These results indicated that the bacterial strain YJT-1 exhibited stronger manganese-oxidizing capability at higher Mn(II) concentrations, thereby reaching a higher BMO content.

### EMBODIMENT 6

In this embodiment, the BMO synthesis ability of the bacterial strain YJT-1 was tested with different carbon sources.

Four Erlenmeyer flasks (500 mL) were prepared, each containing 200 mL of basal inorganic salt liquid culture medium. The carbon source of each culture medium was set to be glucose (1 g/L), D-xylose (1 g/L), sodium L-glutamate (1 g/L), and peptone (0.8 g/L) + yeast extract (0.2 g/L), respectively. The salinity in each culture medium was set at 3.5%. The Mn(II) concentration in each culture medium was set at 100 mg/L. The culture of the bacterial strain YJT-1 in the logarithmic growth phase, which was obtained from step (S4), was inoculated into each sterile medium at 2% (v/v). These Erlenmeyer flasks were incubated at 30°C with shaking at 120 rpm/min for 20 consecutive days. 5 mL of the culture was collected from each Erlenmeyer flask every 4 days, followed by centrifugation, so as to retain precipitates. The BMO content in the precipitates was quantified using the LBB assay. The manganese-oxidizing capability of the bacterial strain YJT-1 was evaluated based on variation in the BMO content.

Each basal inorganic salt liquid culture medium contained 0.4 g/L MgCl₂·6H₂O, 0.754 g/L CaCl₂, 5 g/L KCl, 0.142 g/L Na₂SO₄, 0.085 g/L NaNO₃, and 0.026 g/L K₂HPO₄.

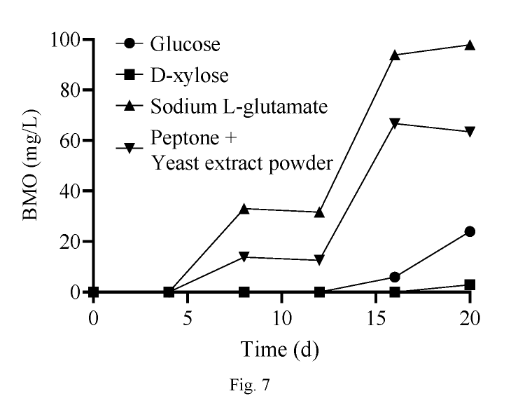

As shown in FIG. 7, the type of the carbon source had a significant impact on the BMO synthesis capability of the bacterial strain YJT-1. When L-glutamate sodium was used as the main carbon source, the BMO content reached a maximum of 97.83 mg/L (for 20 days), which was approximately 4.08 times that with glucose (24.00 mg/L) and approximately 34.44 times that with D-xylose (2.84 mg/L). The BMO content in the presence of complex carbon source (i.e., peptone + yeast extract) was 63.49 mg/L.

### EMBODIMENT 7

In this embodiment, the synergistic effect of carbon source and Mn(II) on the degradation of benzo[a]pyrene (BaP) by the bacterial strain YJT-1 was evaluated.

The bacterial strain YJT-1 obtained in step (S4) was inoculated into 200 mL of inorganic salt liquid culture medium, and was incubated at 30°C with shaking at 120 rpm/min for 24 h to activate the bacterial strain YJT-1. The activated bacterial strain YJT-1 was inoculated into basal inorganic salt liquid culture medium containing 2 mg/L BaP at 2% (v/v). Three groups of experiments were set up, numbered 1, 2 and 3 respectively. Group 1 was a control group with a salinity of 3.5%. Group 2 was also a control group with a salinity of 3.5% and a carbon source of sodium L-glutamate (1 g/L). Group 3 was an experimental group with a salinity of 3.5%, a carbon source of sodium L-glutamate (1 g/L), and a Mn(II) concentration of 100 mg/L. The three groups were incubated at 30°C with shaking at 120 rpm/min for 7 days. 5 mL of the culture was collected from each group at 1 day, 2 days, 3 days, 5 days, and 7 days respectively, and then centrifuged at 10,000 r/min for 10 minutes, so as to obtain a series of supernatants for each group. These supernatants were taken to determine the content of BaP in water and calculate the degradation efficiency of BaP for each group.

The inorganic salt liquid culture medium contained 10 g/L NaCl, 0.4 g/L MgCl₂·6H₂O, 0.754 g/L CaCl₂, 5 g/L KCl, 0.142 g/L Na₂SO₄, 0.085 g/L NaNO₃, 0.026 g/L K₂HPO₄, 0.2 g/L MnSO₄·H₂O, 0.8 g/L peptone, and 0.2 g/L yeast extract.

Each basal inorganic salt liquid culture medium contained 0.4 g/L MgCl₂·6H₂O, 0.754 g/L CaCl₂, 5 g/L KCl, 0.142 g/L Na₂SO₄, 0.085 g/L NaNO₃, and 0.026 g/L K₂HPO₄.

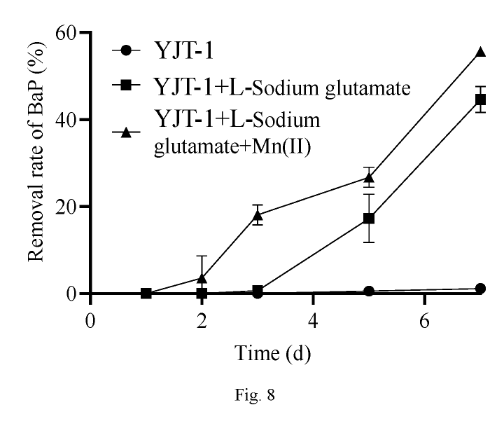

As shown in FIG. 8, the addition of carbon source and Mn(II) significantly improved the degradation efficiency of BaP in water by the salt-tolerant manganese-oxidizing bacterial strain YJT-1. On the 7th day, the degradation efficiency of BaP in water reached 56.10%, which was 33 times and 1.2 times that of Group 1 (1.7%) and Group 2 (46.73%), respectively. Under the synergistic effect of the carbon source and Mn(II), the bacterial strain YJT-1effectively degraded BaP in water.

### EMBODIMENT 8

In this embodiment, the remediation effect of the bacterial strain YJT-1on benzo[a]pyrene-contaminated saline-alkali soil sampled from Xinjiang was evaluated.

The bacterial strain YJT-1 obtained in step (4) was inoculated into 200 mL of inorganic salt liquid culture medium, and was incubated at 30°C with shaking at 120 rpm/min for 24 h to activate the bacterial strain YJT-1. 60 mL of the activated culture was taken from the aforementioned culture medium, and was centrifuged to remove its supernatant, followed by addition of phosphate buffer to suspend it. Then, the bacterial suspension was centrifuged at 10,000 rpm for 10 min to discard its supernatant, repeating this process three times to remove the components of the culture medium. The washed bacterial cells were added to 100 g of saline-alkali soil sampled from Xinjiang containing 1.7 mg/kg BaP (pH: 8.9; organic matter: 32.6 g/kg; Mn(II) content: 14.4 mg/kg; free manganese oxide content: 148.9 mg/kg; and amorphous manganese oxide content: 12.3 mg/kg), homogenized well, transferred to petri dishes, and incubated at a temperature of 30°C and a relative humidity (RH) of 50%. Soil samples were collected after incubation for 1, 3, 7, and 14 days to determine the BaP content in the soil samples and calculate the degradation efficiency of BaP.

The inorganic salt liquid culture medium contained 10 g/L NaCl, 0.4 g/L MgCl₂·6H₂O, 0.754 g/L CaCl₂, 5 g/L KCl, 0.142 g/L Na₂SO₄, 0.085 g/L NaNO₃, 0.026 g/L K₂HPO₄, 0.2 g/L MnSO₄·H₂O, 0.8 g/L peptone, and 0.2 g/L yeast extract.

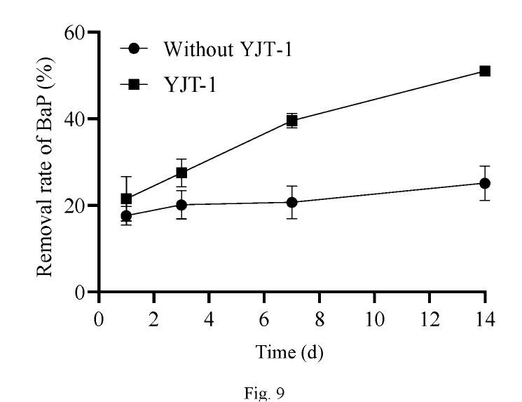

As shown in FIG. 9, the bacterial strain YJT-1 achieved a degradation efficiency of 59.62% for BaP within 14 days in the saline-alkali soil of Xinjiang. During the initial incubation phase, the degradation efficiency of BaP in the soil samples by the bacterial strain YJT-1 was relatively low. On day 3, the degradation efficiency of BaP was 27.74%, possibly because the bacterial strain YJT-1 was still in the stage of adapting to this environment. By day 7, the degradation efficiency of BaP had increased significantly to 44.89%. The bacterial strain YJT-1 continuously removed BaP in the saline-alkali soil, which demonstrated that it had important application value in the remediation of benzo[a]pyrene-contaminated saline-alkali soil.

### EMBODIMENT 9

In this embodiment, the remediation effect of the bacterial strain YJT-1on benzo[a]pyrene-contaminated saline-alkali soil sampled from Ningxia was evaluated.

The bacterial strain YJT-1 obtained in step (S4) was inoculated into 200 mL of inorganic salt liquid culture medium, and was incubated at 30°C with shaking at 120 rpm/min for 24 h to activate the bacterial strain YJT-1. 60 mL of the activated culture was taken from the aforementioned culture medium, and was centrifuged to remove its supernatant, followed by addition of phosphate buffer to suspend it. Then, the bacterial suspension was centrifuged at 10,000 rpm for 10 min to discard its supernatant, repeating this process three times to remove the components of the culture medium. The washed bacterial cells were added to 100 g of saline-alkali soil sampled from Ningxia containing 1.7 mg/kg BaP (pH: 7.85; organic matter: 56.0 g/kg; Mn(II) content: 13.7 mg/kg; free manganese oxide content: 211.0 mg/kg; and amorphous manganese oxide content: 55.7 mg/kg), homogenized well, transferred to petri dishes, and incubated at a temperature of 30°C and a relative humidity (RH) of 50%. Soil samples were collected after incubation for 1, 3, 7 and 14 days to determine the BaP content in the soil samples and calculate the degradation efficiency of BaP.

The inorganic salt liquid culture medium contained 10 g/L NaCl, 0.4 g/L MgCl₂·6H₂O, 0.754 g/L CaCl₂, 5 g/L KCl, 0.142 g/L Na₂SO₄, 0.085 g/L NaNO₃, 0.026 g/L K₂HPO₄, 0.2 g/L MnSO₄·H₂O, 0.8 g/L peptone, and 0.2 g/L yeast extract.

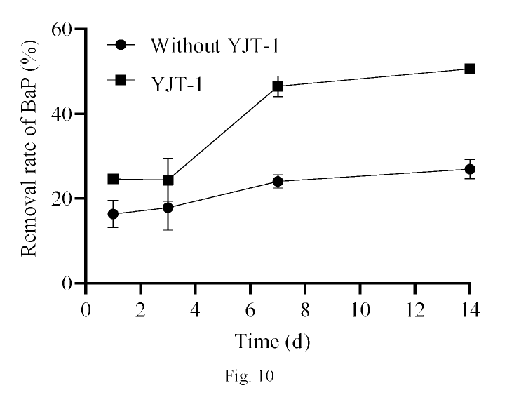

As shown in FIG. 10, the addition of the bacterial strainYJT-1 effectively improved the degradation efficiency of BaP in the saline-alkali soil of Ningxia. Within 14 days, the degradation efficiency of BaP increased from approximately 28% (natural degradation) to approximately 51% (bioenhanced degradation), representing a significant enhancement. The degradation efficiency of BaP, after adding the bacterial strainYJT-1, was much higher than that of the control group without the bacteria strain YJT-1 on day 7 and day 14. During the initial incubation phase, the degradation efficiency of BaP in the soil samples was relatively low. On day 3, the degradation efficiency of BaP was 20.84%, possibly because the bacterial strain YJT-1 was still in the stage of adapting to the environment. By day 14, the degradation efficiency of BaP was greater than 50%.

### EMBODIMENT 10

In this embodiment, the remediation effect of the bacterial strain YJT-1on benzo[a]pyrene-contaminated saline-alkali soil sampled from Shandong was evaluated.

The bacterial strain YJT-1 obtained in step (S4) was inoculated into 200 mL of inorganic salt liquid culture medium, and was incubated at 30°C with shaking at 120 rpm/min for 24 h to activate the bacterial strain YJT-1. 60 mL of the activated culture was taken from the aforementioned culture medium, and was centrifuged to remove its supernatant, followed by addition of phosphate buffer to suspend it. Then, the bacterial suspension was centrifuged at 10,000 rpm for 10 min to discard its supernatant, repeating this process three times to remove the components of the culture medium. The washed bacterial cells were added to 100 g of saline-alkali soil sampled from Shandong containing 1.7 mg/kg BaP (pH: 9.15; organic matter: 25.0 g/kg; Mn(II): 20.70 mg/kg; free manganese oxide: 172.9 mg/kg; and amorphous manganese oxide: 35.8 mg/kg), homogenized well, transferred to petri dishes, and incubated at a temperature of 30°C and a relative humidity (RH) of 50%. Soil samples were collected after incubation for 1, 3, 7, and 14 days to determine the BaP content in the soil samples and calculate the degradation efficiency of BaP.

The inorganic salt liquid culture medium contained 10 g/L NaCl, 0.4 g/L MgCl₂·6H₂O, 0.754 g/L CaCl₂, 5 g/L KCl, 0.142 g/L Na₂SO₄, 0.085 g/L NaNO₃, 0.026 g/L K₂HPO₄, 0.2 g/L MnSO₄·H₂O, 0.8 g/L peptone, and 0.2 g/L yeast extract.

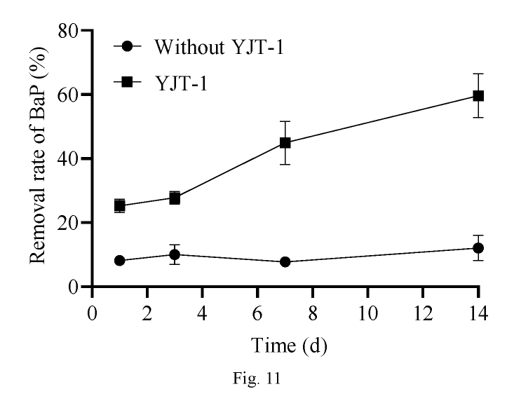

As shown in FIG. 11, the degradation efficiency of BaP in the experimental group with the strain YJT-1 added was consistently and significantly higher than that in the control groups without bacteria added. After adding the bacterial strain YJT-1, the degradation efficiency of BaP in the soil samples was 25% on day 1. As incubation time increased, the degradation efficiency of BaP continuously increased. By day 14, the degradation efficiency of BaP reached 59.6%. This indicated that the bacterial strain YJT-1 effectively adapted to the environment of saline-alkali soil in Shandong and exhibited efficient and continuous degradation capability of BaP.

Collectively, the above embodiments demonstrated that this application successfully isolated the salt-tolerant manganese-oxidizing bacterial strain YJT-1. This strain effectively enhanced BaP degradation in saline-alkali environments. It exhibited strong adaptability to high-salinity and alkaline conditions. The strain maintained high metabolic activity under stress and promoted the formation of BMOs. These BMOs possessed strong catalytic oxidation capability, which facilitated efficient BaP degradation and improved the remediation of polycyclic aromatic hydrocarbon (PAH) contamination in saline-alkali soils.

The embodiments described above are merely specific implementations of this disclosure, and are not intended to limit this disclosure. Any changes or replacements made by those shilled in the art without paying creative effort shall fall with the scope of the present disclosure defined by the appended claims.

What is claimed is:

1. A salt-tolerant manganese-oxidizing bacterial strain YJT-1, wherein the bacterial strain YJT-1 is a Halobacillus sp.strain, which has been deposited in China General Microbiological Culture Collection Center (CGMCC) on March 7, 2025 with an accession number of CGMCC No. 33748.

2. A method of remediating a benzo[a]pyrene-contaminated soil sample or a benzo[a]pyrene-contaminated water sample, comprising:
introducing a bacterial suspension of the bacterial strain YJT-1 of claim 1 into the benzo[a]pyrene-contaminated soil sample or the benzo[a]pyrene-contaminated water sample to degrade benzo[a]pyrene.

3. A method for preparing a biogenic manganese oxide, comprising:
oxidizing divalent manganese ions under the action of the bacterial strain YJT-1 of claim 1 to form the biogenic manganese oxide.

4. A microbial agent for remediating a benzo[a]pyrene-contaminated soil or water sample, comprising:
cells of the bacterial strain YJT-1 of claim 1.
